(19) United States
(12) Patent Application Publication
(10) Pub. No.: US 2026/0041534 A1
(43) Pub. Date: Feb. 12, 2026

(54) ULTRASOUND SYSTEM AND METHOD FOR CLEANING AND/OR REGENERATING ONE OR MORE STRUCTURES INSIDE AN ORAL CAVITY

(71) Applicant: Fraunhofer-Gesellschaft zur Förderung der angewandten Forschung e.V., München (DE)

(72) Inventors: Anas BEN ACHOUR, Dresden (DE); Tom Alexander SCHRÖDER, Dresden (DE); Günter LAUER, Dresden (DE)

(21) Appl. No.: 18/876,851

(22) PCT Filed: Jun. 27, 2023
(86) PCT No.: PCT/EP2023/067488

§ 371 Date: Oct. 8, 2025

(30) Foreign Application Priority Data
Jun. 27, 2022 (DE) 10 2022 115 920.5

(51) Int. Cl. A61C 17/20; A61C 17/22

(52) U.S. Cl. CPC A61C 17/20; A61C 17/228

(57) ABSTRACT

Disclosed herein is an ultrasound system and method for cleaning and/or regenerating one or more structures within an oral cavity. The ultrasound system includes a bite guard for insertion and positioning in an oral cavity. The bite guard includes one or more ultrasound transducers for applying a selected ultrasound signal to one or more structures within an oral cavity to clean and/or regenerate the one or more structures within the oral cavity.

### CROSS-REFERENCE TO RELATED APPLICATION(S)

This application is a national phase of International Patent Application No. PCT/EP2023/067488 filed on Jun. 27, 2023, which claims priority to German Patent Application No. 10 2022 115 920.5 dated Jun. 27, 2022, the contents of both of which are incorporated fully herein by reference.

### TECHNICAL FIELD

Various embodiments relate to an ultrasound system and method for cleaning and/or regenerating one or more structures within an oral cavity.

### BACKGROUND

In general, inadequate dental hygiene may have a significant impact not only on dental health but also on a person's general state of health. Adequate cleaning of the teeth is highly dependent on motivation, continuity and the correct use of toothbrushes and interdental care. There may well be people who are unable to meet these requirements and are therefore dependent on the help of others for daily tooth cleaning.

Despite technical progress, daily tooth cleaning is largely based on the principle of mechanical abrasion. This is usually carried out by manually guided manual toothbrushes in combination with toothpaste. The ingredients of the toothpaste are usually aimed at remineralizing the tooth enamel, inhibiting inflammation and increasing the cleaning performance (e.g. by means of so-called cleaning particles). When cleaning teeth, the technique of the person performing the cleaning, the duration and number of cleaning procedures may have a significant influence on the cleaning result. For example, insufficient removal of impurities (such as a biofilm or an already mineralized biofilm (tartar)) or damage to the enamel due to excessive abrasion forces during brushing in the presence of cleaning particles in the toothpaste. For example, technically supported manual toothbrushes may already partially compensate for these adverse effects by providing direct feedback to the user (e.g. through integrated timers and/or force gauges).

Commercially available (ultra) sonic toothbrushes are generally based on the principle of vibrating a brush head or a specific geometric arrangement of bristles. These may create a relative movement between the bristles and the teeth by vibrating the brush head, which may remove plaque by abrasion and clean the teeth in the same way as the manual brushing technique. Newer ultrasonic toothbrushes may oscillate in a much higher frequency range, which essentially creates a relative movement of the bristles (instead of the entire brush head). The cleaning of the tooth surfaces and gums may be provided by cavitation bubbles in the saliva or toothpaste, which implode as a result of the sound waves.

One disadvantage of such toothbrushes is their design, which essentially corresponds to that of a conventional toothbrush and therefore the cleaning result is heavily dependent on the consistent and correct use of the user. In detail, a consistent sweeping of each tooth is required and the manual guidance of the brush may not be able to achieve an optimal sound wave field in the region to be cleaned. As a result, the cleaning may be insufficient. There is also a risk of pressing the toothbrush too hard or too softly against the teeth, as well as insufficient timing of the brushing process. For example, certain regions may be brushed too long and other regions too short and therefore insufficiently. In addition, conventional (ultrasonic toothbrushes) usually do not have a targeted adjustability of an oscillation frequency in the ultra-frequency region in order to specifically adapt the acoustic field to the needs of a user. Another significant disadvantage of the classic toothbrush design is that the spaces between the teeth are usually insufficiently cleaned. This may generally only be achieved satisfactorily with the additional use of interdental space brushes and/or dental floss.

One of the aims of tooth brushing is to prevent the formation of biofilm and/or tartar on the teeth through regular removal. For example, tartar may develop in several steps: 1. protein adsorption, 2. bacterial adhesion, 3. bacterial biofilm formation and maturation, and 4. mineralization of the mature biofilm (e.g. so-called plaque), which results in the actual tartar. Illustratively, this means that due to a lack of cleaning, the bacterial metabolic products (especially acids) may lead to damage to the tooth substance and thus ultimately to complete tooth loss. The loss of teeth generally leads directly to a reduction in the quality of life of the person affected and, in the case of dentures, causes considerable additional costs.

### SUMMARY

According to various aspects, a device and a method are provided by means of which the daily cleaning of teeth (e.g. a removal of a biofilm and/or of tartar from smooth surfaces and/or from between the teeth) may be improved.

According to various aspects, there is provided a device and a method providing comprehensive functionalities for improving an oral hygiene of a user.

According to various aspects, a device is provided that enables a plurality of predetermined programs for stimulating a regenerative behavior of teeth, a periodontium (e.g., the gums), and a jawbone.

According to various aspects, a device and a method are provided which enable targeted application of ultrasound waves having frequencies in predetermined regions to the oral cavity.

According to various aspects, a device comprising a bite guard (e.g., a mouthpiece) may be provided. According to various aspects, the device may comprise two bite guards. For example, a first bite guard of the two bite guards may be suitable for a maxillary portion of the oral cavity. For example, a second bite guard of the two bite guards may be suitable for a lower jaw portion of the oral cavity.

According to various aspects, a device may be provided comprising a control device (e.g. an electronic control) for controlling the device. For example, the control device may be connected to a bite guard of the device by means of a cable and/or wirelessly (e.g. by radio). In this way, for example, a narrower design may be achieved, which may, for example, increase wearing comfort.

According to various aspects, a device for cleaning the teeth is provided which is not dependent on a manual guide. As a result, sources of error in cleaning may be reduced.

According to various aspects, a device is provided which enables time-synchronous cleaning of all teeth and the interdental spaces corresponding thereto within a region of an oral cavity (e.g. a quadrant) and/or the entire oral cavity.

According to various aspects, the ultrasound system may be used to improve osseointegration of implants (e.g. to promote healing). For example, a bite guard insert adapted to the implants may be inserted into the ultrasound system.

According to various aspects, the ultrasound system may be used to improve healing after bone augmentation. For example, a bite guard insert adapted to an (e.g. operated) anatomical or pathological structure may be inserted into the ultrasound system.

According to various aspects, the ultrasound system may be used to delay (e.g. prevent) bone atrophy.

According to various aspects, there is provided an ultrasound system that may be used in patients with osteoradionecrosis and/or antiresorptive-associated jaw necrosis for cleaning teeth and/or supporting regeneration of structures within the oral cavity.

According to various aspects, the device may comprise an inlet and/or an outlet for introducing a coupling medium (e.g. a liquid and/or a gel and/or a toothpaste) into the oral cavity or removing it from the oral cavity. The inlet and/or the outlet may be coupled to the bite guard, e.g. integrated into the bite guard.

According to various aspects, there is provided an ultrasound system comprising: a bite guard for insertion and positioning in an oral cavity, the bite guard including one or more ultrasound transducers for applying a selected ultrasound signal to one or more structures within an oral cavity (e.g. a plurality of teeth and/or structures adjacent to a plurality of teeth (e.g. directly and/or indirectly), such as corresponding interdental spaces, a periodontium, adjacent tissue (e.g. gums)) for cleaning and/or regenerating the one or more structures within the oral cavity.

Illustratively, various types of soiling may be removed using ultrasound waves. In particular, light deposits (e.g. biofilms, food residues), stubborn impurities (e.g. tartar) and, in general, bacterial impurities may be removed from teeth, the corresponding interdental spaces and the gums by using oscillations in different, predetermined frequency spectra.

For example, the ultrasound transducers (e.g. the ultrasound emitters) mounted in the bite guard may clean a human and/or animal dentition completely, time-synchronously and gently by means of a targeted alignment of an acoustic field (e.g. an ultrasound (wave) field). For example, the bite guard may be designed in such a way that it may be easily adapted to the human and/or animal dentition (e.g. an individual tooth position). This may, for example, make it more comfortable to wear and/or allow the system to be used by several users. For example, it may be possible to clean the teeth (e.g. the tooth surfaces), the corresponding interdental spaces and the gums without the use of brushes (or bristles).

For example, a bite guard of the ultrasound system may have a communication interface (e.g. for wired and/or wireless communication). This allows the bite guard to be connected to a control device (e.g. a device configured for this purpose and/or a device on which a corresponding control app is configured).

For example, the control device may be configured to support the user during use with audiovisual signals (e.g. an audiovisual toothbrushing guide). For example, the ultrasound system may be protected from dust and/or liquids to enable fast cleaning. For example, the ultrasound system may enable low-noise use. For example, the ultrasound system may comprise a dental splint that may be adapted to the teeth (e.g. a bite guard insert) and thus enable a high level of comfort for a user. For example, the ultrasound system may comprise separate bite guards for an upper row of teeth and a lower row of teeth. For example, the adaptable dental splint (e.g. adaptable to a bite shape) may be configured for insertion into the bite guard. A modular design thus makes it possible to replace the bite guard. This makes it possible, for example, to clean contact surfaces where the ultrasound system comes into contact with the dentition. Furthermore, this enables hygienic use by several people, as the dental splints may be inserted individually.

According to various aspects, an ultrasound system may be provided for cleaning and/or regenerating one or more structures within an oral cavity. The one or more structures within the oral cavity may comprise (e.g., may be) a plurality of teeth and/or structures directly or indirectly adjacent to the plurality of teeth.

### BRIEF DESCRIPTION OF THE DRAWINGS

Various exemplary embodiments are shown in the figures and are explained in more detail below.

FIGS. 1 to 9 each show an ultrasound system according to different aspects.

FIG. 10 shows a method for cleaning and/or regenerating one or more structures within an oral cavity according to various aspects.

### DESCRIPTION

In the following detailed description, reference is made to the accompanying drawings which form part thereof and in which specific embodiments in which the invention may be practiced are shown for illustrative purposes. In this regard, directional terminology such as “top”, “bottom”, “front”, “rear”, “forward”, “rearward”, etc. is used with reference to the orientation of the figure(s) described. Since components of embodiments may be positioned in a number of different orientations, the directional terminology is for illustrative purposes and is not limiting in any way. It is understood that other embodiments may be used and structural or logical changes may be made without departing from the scope of protection of the present invention. It is to be understood that the features of the various exemplary embodiments described herein may be combined with each other, unless specifically indicated otherwise. The following description is therefore not to be construed in a limiting sense, and the scope of protection of the present invention is defined by the appended claims.

According to various aspects, one or more ultrasound waves may be provided (e.g., generated). It is understood that the one or more ultrasound waves may be represented by an ultrasound signal corresponding to the respective ultrasound wave of the one or more ultrasound waves. Therefore, an ultrasound wave may also be referred to herein by means of the ultrasound signal corresponding to and vice versa. An ultrasound wave may be understood as the actual physical (sound) shaft that may propagate within a medium or a substance. An ultrasound signal may be understood as a metrological and/or excitation-related equivalent of an ultrasound wave, e.g. in the form of one or more characteristics of the ultrasound wave (e.g. as a measured value).

Each ultrasound signal may comprise information (e.g. in the form of data) representing a corresponding ultrasound wave, such as: an intensity, and/or a frequency, and/or a phase length, and/or a wavelength, and/or a duration (e.g. a time delay), and/or a start time, and/or an end time of a corresponding ultrasound wave. Furthermore, ultrasound signals may be pulsed. Illustratively, a pulsed ultrasound signal may generate multiple ultrasound waves per pulse, comprising one or more predetermined frequencies. There may be a pause between two pulses during which no ultrasound waves are generated. A number of pulses per unit of time (e.g. per second) may be referred to as the pulse frequency. It is understood that the pulse frequency represents the time between a respective start of two directly successive ultrasound pulses.

For example, the ultrasound shaft corresponding to the ultrasound signal may be generated on the basis of an ultrasound signal (e.g. by means of an ultrasound transducer (e.g. an ultrasound (wave) emitter)). For example, the ultrasound signal corresponding to the ultrasound wave may be generated (e.g. determined, measured, detected) on the basis of an ultrasound shaft (e.g. by means of an ultrasound transducer (e.g. by means of an ultrasound (wave) receiver)). Coupling an ultrasound signal into a medium or a substance” may be understood to mean that an ultrasound wave is generated in the medium or in the substance which corresponds to the ultrasound signal.

An ultrasound transducer may be defined herein as a component that may convert ultrasound waves into ultrasound signals and vice versa. For example, an ultrasound transducer may be configured to emit one or more ultrasound signals in the form of one or more ultrasound waves into a medium. Such an ultrasound transducer may, for example, comprise (e.g. consist of) one or more ultrasound emitters. For example, an ultrasound transducer may be to receive one or more ultrasound waves within a medium and/or from a medium and to convert the received one or more ultrasound waves into a corresponding ultrasound signal. Such an ultrasound transducer may, for example, comprise (e.g. consist of) one or more ultrasound receivers. It is understood that an ultrasound transducer may comprise one or more ultrasound receivers as well as one or more ultrasound emitters.

For example, one or more ultrasound waves may be generated from one or more ultrasound signals by means of one or more ultrasound transducers (e.g. by means of one or more ultrasound emitters). For example, one or more ultrasound waves may be generated based on the piezoelectric effect, e.g. by means of a piezoelectric material or a piezoelectric crystal. For example, one or more ultrasound waves may be generated from one or more corresponding ultrasound signals by means of the piezoelectric effect. For example, one or more ultrasound waves may be received as one or more corresponding ultrasound signals by means of one or more ultrasound transducers (e.g. by means of one or more ultrasound receivers). For example, the one or more received ultrasound waves may be converted into one or more corresponding ultrasound signals. Generating ultrasound signals or determining ultrasound signals may be understood to mean a process that may comprise receiving (e.g. capturing) one or more ultrasound waves and converting the received one or more ultrasound waves into the corresponding ultrasound signals. For example, one or more ultrasound transducers may be configured to determine ultrasound signals. For example, ultrasound waves may be converted into ultrasound signals based on the piezoelectric effect, e.g. by means of a piezoelectric material or a piezoelectric crystal.

The impedance, i.e. the resistor that counteracts the propagation of shafts, may be important for the propagation (e.g. a speed, a segment) of ultrasound waves in a material. In particular, an ultrasound wave may be (at least) partially deflected (e.g. reflected) and/or partially transmitted at interfaces between two neighboring substances. The respective deflected or transmitted portion depends on an impedance difference at the interface of the two neighboring materials.

The greater the impedance difference at the interface of the two adjacent materials, the greater the proportion of the deflected ultrasound wave may be (compared to the transmitted proportion). At an interface of two substances with a large impedance difference (e.g. more than 25 %, 50 %, 75 % or more than 100 %), an incident ultrasound wave may be strongly deflected (e.g. more than 10 %, 25 %, 50 %, 75 % or more than 95 %). For example, this difference may be particularly pronounced between a gas (e.g. air) and a liquid (e.g. water).

The smaller impedance difference at the interface of the two adjacent substances, the smaller (compared to the transmitted portion) the deflected portion of the ultrasound wave may be. Furthermore, due to the high impedance difference at the transition from a first material to a second material of the two adjacent materials, a direction of travel of the deflected portion of the ultrasound wave may be changed more strongly than with a smaller impedance difference.

According to various aspects, a coupling medium may be used to prevent the formation of air bubbles. For example, the coupling medium may be used to fill a cavity filled with a gas. Thus, ultrasound waveguiding may be enabled within the cavity. For example, the coupling medium may be configured to reduce an intensity of an ultrasound wave from the coupling medium to less than 1%, with a simple travel distance of 6 cm. For example, the coupling medium may be water and thus reduce the intensity of the ultrasound wave by 50% over a distance of 6 m. For example, the coupling medium may have an absorption coefficient of ultrasound waves of 1 dB/m or less. It is understood that the absorption of ultrasound waves is frequency-dependent and the values mentioned refer to the frequency range relevant for an application. The lowest possible absorption of the coupling medium may enable optimum energy consumption of the ultrasound system.

According to various aspects, the coupling medium may comprise one or more additives, such as additional ingredients suitable to promote regeneration processes (e.g., remineralization processes) (e.g., the coupling medium may comprise fluorine and/or hydroxyapatite and/or zinc oxide for this purpose) and/or to promote cleaning processes (e.g., the coupling medium may comprise cleaning agents for this purpose). Cleaning agents may be additives of a toothpaste which, together with a toothbrush, may remove impurities (e.g. plaque and/or harmful bacteria) from a tooth surface. According to various aspects, the cleaning bodies in a coupling medium may be adapted to one or more ultrasound waves, e.g. by supporting cavitation effects at certain frequencies.

According to various aspects, a transit time of an ultrasound wave may be determined. An ultrasound wave may be emitted at a starting point. The ultrasound wave propagates within a medium at a propagation speed (e.g. at the speed of sound). The propagation speed may depend on the medium. The propagation speed may be represented by the impedance, for example. The propagation time may be determined, for example, on the basis of the propagation speed of the ultrasound wave within the medium and the propagation time.

According to various aspects, one or more ultrasound signals may be provided (e.g. generated). Ultrasound may refer to one or more (sound) shafts, each having a frequency between 20 kHz and 10 GHz. For example, an ultrasound wave may have (e.g. have) a frequency between 1 and 40 MHz. For example, an ultrasound wave may have an intensity (e.g. radiation intensity, sound intensity) between 5 mW/cm² and 125 mW/cm².

According to various aspects, an ultrasound system is provided, which may comprise one or more functionalities. The respective functionalities of the one or more functionalities may be based on physical effects of ultrasound. For example, the one or more functionalities may comprise (e.g. be) one or more of the following functionalities. In particular, the respective functionalities of different ultrasound signals may depend on a certain combination of ultrasound frequencies, intensities and pulse frequencies of the ultrasound signals (or the corresponding ultrasound waves), as described below.

For example, one of the one or more functionalities may be a cleaning of the tooth surfaces (e.g. a removal of biofilm and/or tartar) by means of a cavitation effect. For example, the cavitation effect may be improved by a suitable coupling medium (e.g. a suitable toothpaste). Furthermore, the cleaning effect may be enhanced by a combination effect with the coupling medium, e.g. due to cleaning particles within the coupling medium.

For example, one of the one or more functionalities may be cleaning of the interdental spaces. For example, biofilms, tartar and other impurities may be removed using cavitation.

For example, one of the one or more functionalities may be an antibacterial surface treatment. For example, disinfection may be carried out by ultrasound and/or by light (e.g. ultraviolet light).

For example, disinfection may be carried out by using physical effects. For example, ultrasound waves comprising frequencies between 1 kHz and 500 kHz, e.g. 5 kHz and 300 kHz or 20 kHz and 200 kHz or 20 kHz and 60 kHz may be used. For example, several ultrasound waves with frequencies different from each other may be superposed in order to carry out more effective disinfection. Furthermore, such ultrasound waves may have intensities between 1 mW/cm² and 100 W/cm² or, for example, between 5 mW/cm² and 50 W/cm², or, for example, between 0.1 W/cm² and 10 W/cm². For example, a smaller frequency range and/or lower intensities may consume less energy and achieve gentler cleaning. For example, an application duration may be in a region of one or more minutes. For example, an application duration may be less than 10 min (e.g. less than 5 min, e.g. less than 2 min).

In one example, several ultrasound waves may be used for disinfection, each of which may have a frequency from a frequency range between 20 kHz and 60 kHz. For example, different ultrasound waves have different frequencies. For example, the one or more ultrasound waves may each have a power of approximately 100 W (e.g. 100 W/cm²).

For example, disinfection may be carried out by using chemical effects. For example, ultrasound waves with frequencies between 50 kHz and 5 MHz or, for example, 100 kHz and 2 MHz or, for example, between 200 kHz and 1 MHz may be used for this purpose. For example, several ultrasound waves with different frequencies may be superposed in order to carry out more effective disinfection. Furthermore, such ultrasound waves may have intensities between 1 mW/cm² and 100 W/cm² or, for example, between 5 mW/cm² and 50 W/cm² or, for example, between 0.1 W/cm² and 10 W/cm². For example, an application duration may be in a region of one or more minutes. For example, an application duration may be less than 10 min (e.g. less than 5 min, e.g. less than 2 min).

For example, one of the one or more functionalities may be a stimulation of a regeneration behavior of structures within an oral cavity (e.g. of one or more teeth (e.g. by a remineralization), of a periodontium (e.g. of the gums) and/or of a jawbone (e.g. due to a stimulation of a cell proliferation) due to ultrasound-based stimulation.

For example, pulsed and/or continuously emitted (i.e. non-pulsed) ultrasound waves at certain frequencies may be suitable to stimulate osteoblast proliferation (which may lead to faster bone fracture healing).

Continuously emitted ultrasound waves for stimulating osteoblast proliferation may have frequencies between 30 kHz and 60 kHz or, for example, between 40 kHz and 50 kHz or, for example, of 45 KHz. Continuously emitted ultrasound waves for stimulating osteoblast proliferation may have a power of between 10 mW/cm² and 40 mW/cm² or, for example, between 20 mW/cm² and 30 mW/cm² or, for example, 25 mW/cm².

Pulsed ultrasound waves for stimulating osteoblast proliferation may have frequencies between 45 kHz and 2 MHz or may have frequencies between 500 kHz and 1.5 MHz, for example, or may have frequencies of 1 MHz, for example. Pulsed ultrasound waves for stimulating osteoblast proliferation may have a power of between 100 mW/cm² and 400 mW/cm² or, for example, between 200 mW/cm² and 300 mW/cm² or, for example, 250 mW/cm². Pulsed ultrasound waves for stimulating osteoblast proliferation may have a pulse frequency between 50 Hz and 100 Hz or, for example, between 60 Hz and 70 Hz or, for example, 63 Hz. Pulsed ultrasound waves for stimulating osteoblast proliferation may have a pulse duration of between 1 ms and 20 ms or, for example, between 1 ms and 5 ms or, for example, of around 3 ms (e.g. 3.2 ms).

For example, ultrasound waves with certain frequencies may be suitable for stimulating proliferation of odontoblast-like cells, for example by stimulating the production of proliferation-associated proteins (e.g. hsp25/27, TGFb1, VEGF). For example, pulsed ultrasound waves may be used to stimulate proliferation of odontoblast-like cells from the LIPUS (Low Intensity Pulsed UltraSound) region.

Ultrasound waves from the LIPUS region may have frequencies between 0.5 MHz and 2 MHz, for example between 1 MHz and 1.5 MHz. Ultrasound waves from the LIPUS region may a power between 1 mW/cm² and 1000 mW/cm², for example between 10 mW/cm² and 750 mW/cm² or, for example, between 20 mW/cm² and 500 mW/cm². Ultrasound waves from the LIPUS region may have a pulse frequency between 0.1 kHz and 10 kHz, for example between 0.5 kHz and 5 kHz or, for example, of 1 kHz. Ultrasound waves from the LIPUS region may have a pulse duration of between 1 μs and 1 ms, for example between 50 μs and 500 μs or, for example, of around 200 μs.

For example, pulsed ultrasound may be used to accelerate periodontal ligament regeneration (e.g. a tissue that attaches the tooth to a corresponding bone) after periodontal aponeurotomy (PA) flap surgery.

Pulsed ultrasound waves for accelerating periodontal ligament regeneration may have frequencies between 100 kHz and 5 MHz, for example between 500 kHz and 2 MHz, or may have frequencies of 1.5 MHz, for example. Pulsed ultrasound waves for accelerating periodontal ligament regeneration may have a power of between 1 mW/cm² and 500 mW/cm², for example between 10 mW/cm² and 100 mW/cm² or, for example, 30 mW/cm². Pulsed ultrasound waves for accelerating periodontal ligament regeneration may have a pulse frequency between 0.1 kHz and 10 kHz, for example between 0.5 kHz and 5 kHz or, for example, 1 kHz. Pulsed ultrasound waves for accelerating periodontal ligament regeneration may have a pulse duration of between 0.1 ms and 10 ms, for example between 0.5 ms and 5 ms or, for example, around 3 ms.

For example, pulsed ultrasound may be used to enable better osseointegration of implants. For example, pulsed ultrasound waves may be used to enable better osseointegration of implants from the LIPUS (Low Intensity Pulsed UltraSound) region.

For example, ultrasound waves with certain frequencies may be suitable for enabling remineralization of the dentin using high-intensity focused ultrasound (HIFU).

Ultrasound waves from the HIFU region may have frequencies between 0.5 MHz and 10 MHz, for example between 1 MHz and 5 MHz. Ultrasound waves from the HIFU region may have a power of between 0.5 kW/cm² and 20 kW/cm², for example between 1 kW/cm² and 10 kW/cm² or for example of around 5 kW/cm² (e.g. with a total irradiation time of less than 3 seconds). Due to such a high energy density and the associated temperature increase, ultrasound waves from the HIFU region should only be applied over very short periods of time (e.g. less than 1 ms or e.g. less than 10 μs or e.g. less than 1 μs).

For example, ultrasound waves from the HIFU region may be suitable for remineralizing teeth, in particular the respective tooth surfaces. Remineralization may be supported in particular by a coupling medium comprising fluoride and/or hydroxalapatite.

According to various aspects, provided herein are methods and devices for cleaning and/or regenerating one or more structures within an oral cavity. As used herein, one or more structures within an (e.g., the) oral cavity may be understood to be one or more teeth and/or structures adjacent to the plurality of teeth. These adjacent structures may include, for example, structures of the periodontium (such as gums, jaw bones (e.g., mandibular bone, maxillary bone)), nerves, and/or other structures that are in direct or indirect contact with one or more teeth. Furthermore, the one or more structures within the oral cavity also include surfaces associated with the respective structures (e.g. tooth surfaces, bone surfaces, gum surfaces) and/or spaces (e.g. interdental spaces, interdental spaces, a chamber between the gum and the neck of the tooth, etc.) between respective directly adjacent structures of the one or more structures within the oral cavity.

According to various aspects, there is provided an ultrasound system configured to determine contaminants and/or pathological changes. For example, the ultrasound system may determine whether caries is present, for example by means of light transmission and/or photo-optical transillumination (e.g. fiber optic transillumination). For example, the ultrasound may determine whether tartar and/or periodontal disease is present. According to various aspects, the ultrasound system may be configured to select an ultrasound signal suitable for the determined result in order to remove the determined contamination and/or to support a regeneration process.

According to various aspects, the ultrasound system may be suitable (e.g. used) to improve (e.g. accelerate) regeneration after dental trauma and/or jawbone fractures. For example, a customizable dental splint may be configured to be regularly adjusted to the respective regeneration condition.

It should be understood that the methods described herein are not intended to be therapeutic treatment methods or diagnostic methods per se. However, the methods and components described herein may be used to obtain measurement data and, under certain circumstances, knowledge about a human or an animal. The aspects described herein may be used to support treatment, but are not intended to capture treatment or diagnosis as such.

In the following, various exemplary embodiments of an ultrasound system according to various aspects are explained with reference to the attached figures.

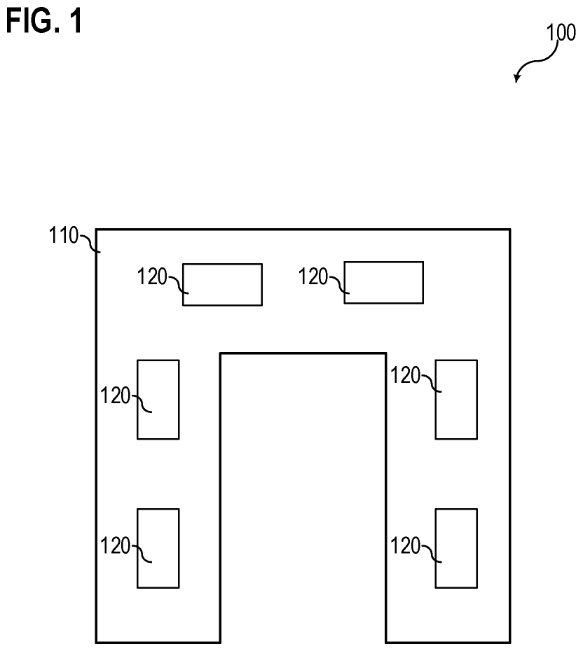

FIG. 1 schematically illustrates an ultrasound system 100 according to various aspects. The ultrasound system 100 may comprise a bite guard 110 configured for insertion and positioning in an oral cavity of a human and/or an animal. Illustratively, such a bite guard is adapted to geometric dimensions of a human and/or animal oral cavity. In particular, the bite guard may be adapted to the geometric dimensions of oral cavities of adults, adolescents and/or children.

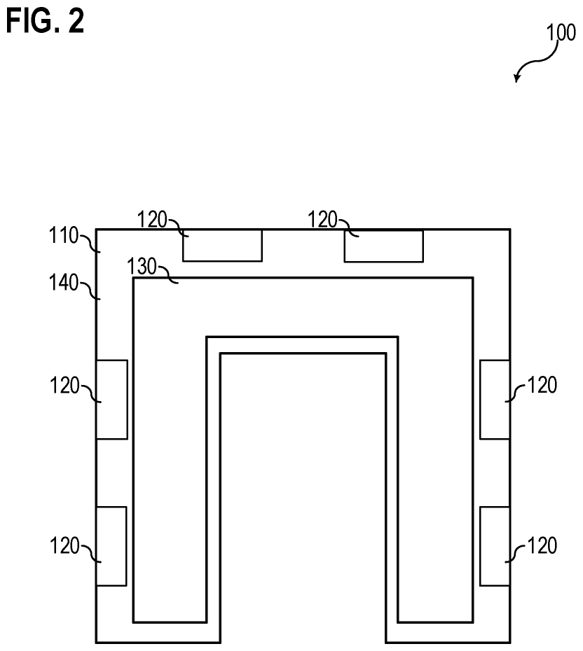

FIG. 2 schematically illustrates an ultrasound system 100 according to various aspects comprising a bite guard insert 130, which is/may be adapted to a tooth shape of a user. According to various aspects, the bite guard 110 may comprise a bite guard cover 140 and the bite guard insert 130. For example, the bite guard insert 130 may comprise (e.g., consist essentially of) a thermoplastic material such that its surface may be conformed to a dental impression of a user. For example, the bite guard insert 130 may be heated, and then the user's dental impression may be created. In connection, when the bite guard insert 130 has cooled down, it may represent the user's dental impression in a dimensionally stable manner. Thus, a minimum distance between the bite guard insert 130 (and thus the bite guard 110) and the user's teeth may be enabled, whereby an effect of the ultrasound waves (e.g. cleaning effect, regeneration effect) may be enhanced.

Further, the bite guard insert 130 allows multiple users to divide an ultrasound system 100 according to various aspects, for example, in which each user has their own bite guard insert 130.

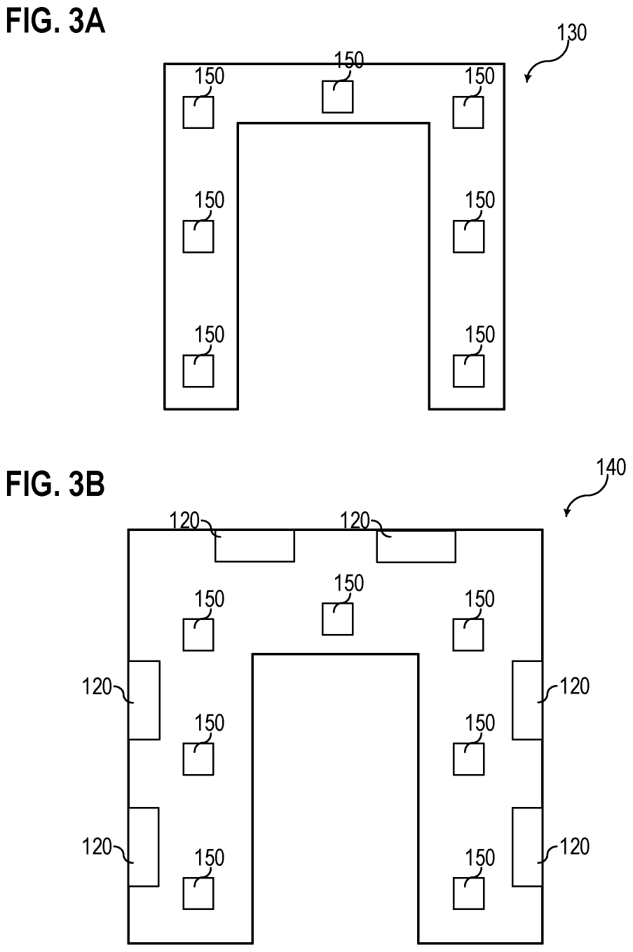

FIGS. 3A and 3B schematically illustrate an ultrasound system 100 according to various aspects, wherein the bite guard insert 130 (FIG. 3A) has been removed from the bite guard cover 140 (FIG. 3B). According to various aspects, the ultrasound system 100 may comprise a plurality of one or more attachment elements 150 configured to attach the bite guard insert 130 to the bite guard cover 140. For example, each of the one or more attachment elements 150 may comprise a first portion and a second portion corresponding to the first portion. For example, the first portion may be attached to the bite guard insert 130 and the second portion may be attached to the bite guard cover 140. For example, the first and second portions of the one or more attachment elements 150 may adhere to each other by means of a magnetic force. For example, the first and second portions of the one or more attachment elements 150 may adhere to each other by means of a mechanical force (e.g., due to a mating mechanism). For example, the one or more attachment elements 150 may comprise a fixing element for fixing the bite guard insert 130 in the bite guard cover 140.

According to various aspects, the bite guard 110 may include one or more ultrasound transducers 120 (e.g., ultrasound emitters) for applying one or more ultrasound signals to one or more structures within an oral cavity. The plurality of ultrasound signals may be suitable for cleaning and/or regenerating the one or more structures within the oral cavity. Here, the respective specific suitability of one of the plurality of ultrasound signals may be determined by its frequency, intensity and/or pulse rate (i.e. if it is a pulsed ultrasound signal).

More specifically, the ultrasound transducers 120 may be configured to convert a predetermined ultrasound signal (e.g., provided by a control device) into a plurality of ultrasound waves. Thus, when the ultrasound system is positioned in an oral cavity, an ultrasound wave field may be generated. Within this field, strong pressure fluctuations may occur, which in turn may trigger cavitations, i.e. the forming and resolution of vapor-filled cavities (illustratively vapor bubbles) in liquids (e.g. in the coupling medium). Cavitation may create a mechanical cleaning effect on solid (e.g. non-liquid, non-gaseous) neighboring structures. In the oral cavity, for example, food residues, biofilms, tartar or other impurities may be loosened from teeth (i.e. tooth surfaces and/or interdental spaces). In an analogous way, impurities from other structures within the oral cavity that are not teeth (e.g. from the gums) may also be loosened and thus removed.

According to various aspects, different ultrasound signals may be provided for different contaminants from each other, i.e. ultrasound signals that differ in their respective ultrasound frequencies, intensities and/or pulse properties.

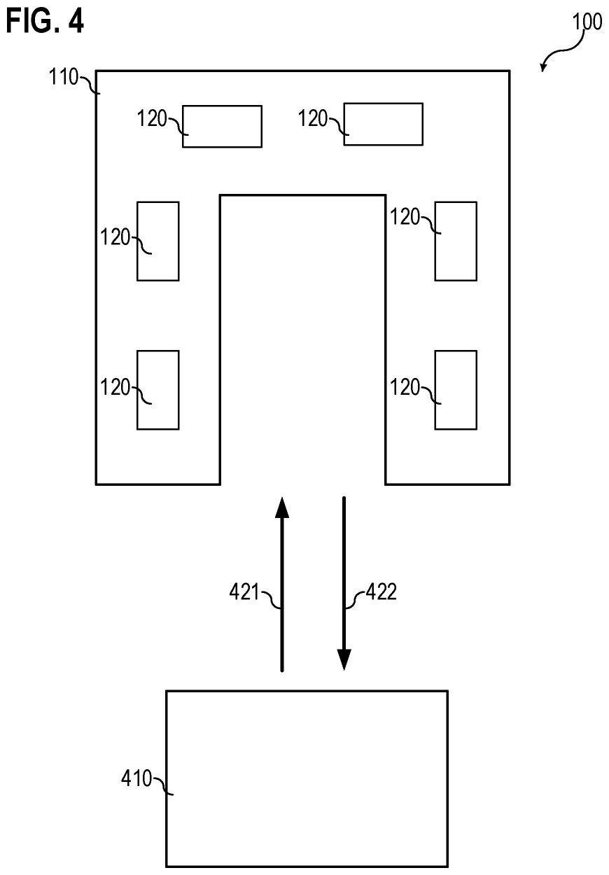

FIG. 4 schematically illustrates an ultrasound system 100 according to various aspects, comprising a control device 410 for controlling the ultrasound system 100.

According to various aspects, the control device 410 may be configured to transmit signals to the bite guard 110, in particular to the ultrasound transducers therein, shown by the arrow 421, and/or to receive signals from the bite guard 110, shown by the arrow 422. For example, the control device 410 may comprise a wired connection and/or a wireless connection for transmitting signals to the bite guard and/or receiving signals from the bite guard 110. It will be understood that in the case of a wireless connection, the bite guard and the control device each comprise a transceiver unit. It is further understood that in a wireless connection, the control device 410 and the bite guard 110 each comprise a separate energy storage unit (e.g. in the form of an accumulator, a battery). In the case of a wired connection, the bite guard 110 and the control device 410 may divide a common energy storage unit.

According to various aspects, the control device 410 may be realized by an app installed, for example, on a mobile device (e.g. a smartphone, tablet, laptop, etc.). This may, for example, be coupled to the bite guard 110 by means of a wireless connection (e.g., NFC, Bluetooth, WLAN, infrared, etc.). However, it is understood that the control device 410 may also be a separate individual device.

According to various aspects, the control device 410 may comprise a memory on which a plurality of ultrasound signals different from each other are stored. The control device 410 may be configured to transmit a respective selected one of the plurality of different ultrasound signals to the bite guard 110, in particular to the one or more ultrasound transducers 120, in response to a user selection. The bite guard 110 may be configured to receive the ultrasound signal transmitted by the control device 410 and forward it to the one or more ultrasound transducers 120. The one or more ultrasound transducers may be configured to convert the received ultrasound signal into a corresponding ultrasound wave and thus generate an ultrasound wave field within the oral cavity.

It is understood that the sound may be stopped in an analog manner and/or another signal may be selected.

Furthermore, it is understood that a series (e.g. a series, a selection) of several ultrasound signals of the several ultrasound signals that are different from each other may also be selected. Thus, for example, a complete cleaning program and/or regeneration program may be enabled.

According to various aspects, the control device 410 may be configured to independently select a suitable ultrasound signal from the plurality of mutually different ultrasound signals.

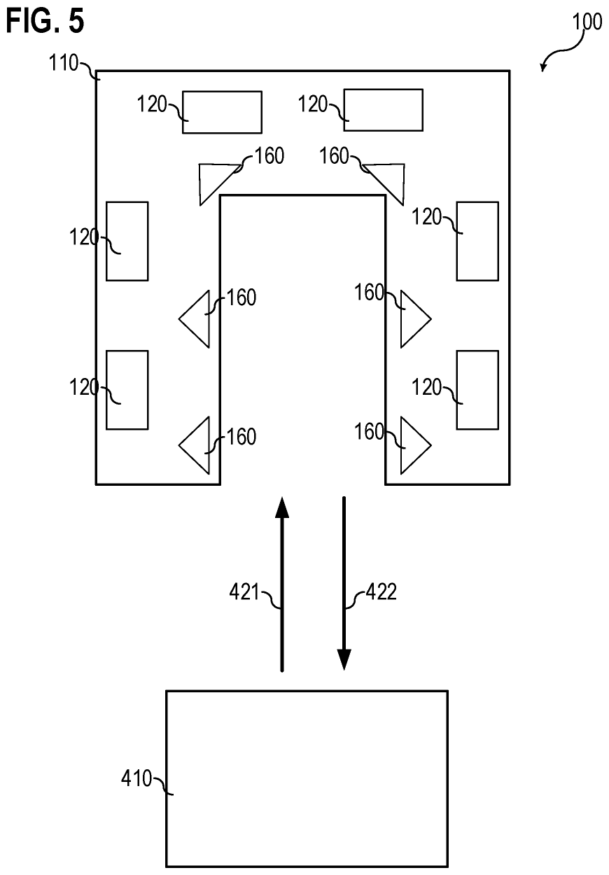

FIG. 5 schematically illustrates an ultrasound system 100 according to various aspects comprising one or more sensors 160. The one or more sensors may be configured to determine one or more properties of the one or more structures within the oral cavity. For example, a property may be: a presence of a contaminant, a degree (e.g., a characteristic) of a contaminant, a type of a contaminant (e.g., tartar, food debris, biofilm, other bacterial contaminants), a presence of a pathological change (e.g., caries, demineralization, bleeding, etc.), a degree of pathological change, etc., and so on.

The one or more sensors 160 may be configured to emit a predetermined sensor signal and subsequently receive it as a measurement signal. The received measurement signal may differ from the emitted sensor signal. The difference may represent one, more, or all of the one or more properties of the one or more structures within the oral cavity, which may be determined by evaluating the difference using the control device 410.

For example, the one or more sensors 160 may comprise (e.g., be) ultrasound receivers configured to receive ultrasound waves emitted by the ultrasound transducers 120 and convert them into a measurement signal. The measurement signal may represent one, more or all of the one or more properties of the one or more structures within the oral cavity. For example, one or more predetermined sensor ultrasound signals may be provided (e.g., stored) in the control device for transmission to the one or more ultrasound transducers 120. The one or more predetermined sensor ultrasound signals may, for example, be suitable for determining certain properties. For example, a first sensor ultrasound signal may be suitable for determining a presence of a contaminant (e.g., tartar). For example, a second sensor ultrasound signal may be suitable for determining a degree of pathological change (e.g. periodontitis). For example, the first and second sensor ultrasound signals may differ from each other. Thus, the ultrasound signal emitted by the one or more ultrasound transducers 120 may be the emitted transmission signal and the signal received by the one or more sensors 160 may be the measurement signal (i.e., the received sensor signal).

For example, the one or more sensors may comprise (e.g. be) one or more optical sensors, such as transmission sensors and/or reflection sensors. The one or more optical sensors may be configured to emit an optical sensor signal and subsequently receive an optical measurement signal. For example, the one or more optical sensors may be configured to emit a light with a specific wavelength. For example, the emitted light may be reflected at and/or transmitted through a surface (e.g. a respective surface of one or more structures within the oral cavity). In connection, the reflected and/or transmitted light may be received by one of the one or more optical sensors (e.g. another (in the case of reflection, transmission) or the same (in the case of reflection). The received light signal may be converted by the sensor into a measurement signal that may represent one, more or all of the one or more properties of the one or more structures within the oral cavity. It will be understood that in combination with a previously described bite guard insert 130, the bite guard insert 130 is permeable to a signal emitted and received by the one or more sensors 160 at the portions corresponding to the one or more sensors 160.

For example, the measurement signal received by the one or more sensors 160 may be transmitted from the one or more sensors 160 to the control device 410, shown by the arrow 422. The control device may be configured to determine the one, the plurality, or all of the one or more properties of the one or more structures within the oral cavity based on the measurement signal. Further, the control device 410 may be configured to select, based on the determination result, a corresponding ultrasound signal or a series of corresponding ultrasound signals that are most suitable for cleaning and/or regeneration and transmit it to the bite guard 110, in particular to the one or more ultrasound transducers 120.

According to various aspects, the control device 410 may be configured to suggest to a user the determination result and the ultrasound signal(s) or series of ultrasound signal(s) corresponding to the determination result. In connection, the user may select an ultrasound signal or a series of ultrasound signals, which are then transmitted by the control device 410 to the bite guard 110, in particular to the ultrasound transducers 120. In this way, the cleaning process or the regeneration process may be adapted to the user's needs.

According to various aspects, a cleaning process and/or a regeneration process may be enhanced due to a coupling medium. For example, the coupling medium may provide better ultrasound wave conduction between the one or more ultrasound transducers 120 and the structures within the oral cavity (e.g., because no gas bubbles are present). For example, the coupling medium may comprise one or more additives that may support a cleaning process (e.g., cleaning agents) and/or may support a regeneration process (e.g., fluoride, hydroxyapatite, zinc oxide (ZnO)).

For example, the coupling medium may be applied to the bite guard before use. However, this carries the risk of (local) overdosing or underdosing by a user.

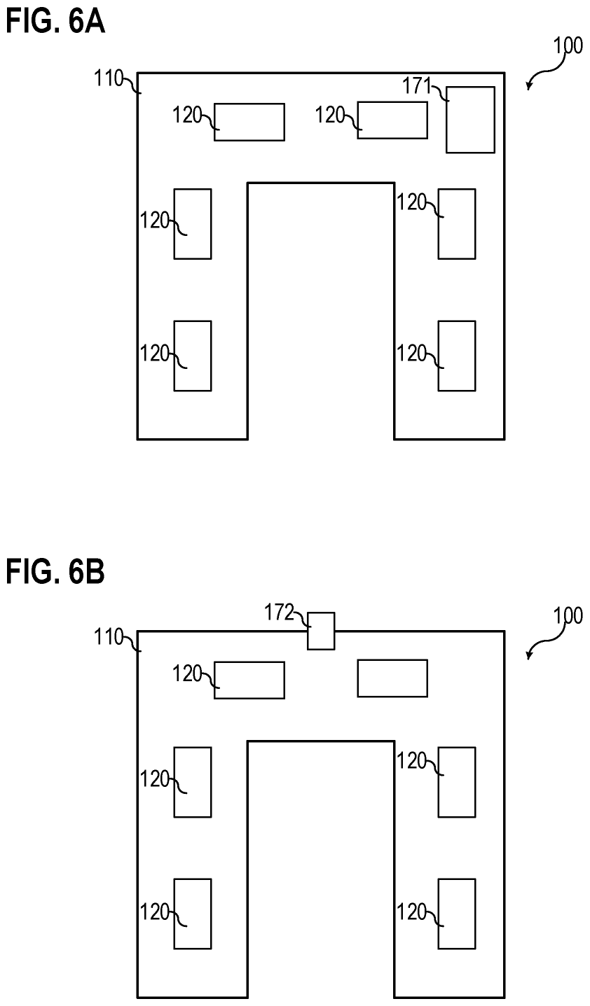

FIG. 6A schematically illustrates an ultrasound system 100 according to various aspects comprising a coupling medium storage 171 for storing a coupling medium. The coupling medium storage 171 may be configured to dispense a certain amount of the coupling medium. Furthermore, the coupling medium storage 171 may be configured to signal to the user when the fill level falls below a predetermined value and/or the coupling medium storage 171 is completely empty. For example, the coupling medium reservoir 171 may be controlled by the control device 410. For example, the control device 410 may be configured to transmit, together with the ultrasound signal, a dosing signal for the coupling medium reservoir 171 to the bite guard 110. The dosing signal may comprise information for the coupling medium reservoir 171 as to how much of the coupling medium is to be dispensed and over what period of time. For example, the dosing may be adapted by the control device 410 to the determined property or properties of the one or more structures within the oral cavity. Further, the control device 410 may be configured to signal the user when the coupling medium reservoir 171 is empty or has less than a predetermined fill level. For example, the bite guard 110 may comprise one or more coupling medium distribution lines to evenly distribute the coupling medium within the bite guard 110. In this way, a continuous supply of coupling medium that is individually adapted to the needs of a user may be ensured.

FIG. 6B schematically illustrates an ultrasound system 100 according to various aspects comprising a coupling medium supply element 172 for supplying a coupling medium. The coupling medium supply element 172 may be designed analogous to the coupling medium storage 171, whereby here the coupling medium is supplied from outside into the bite guard 110. For example, a separate coupling medium reservoir may be provided for this purpose, outside the bite guard 110, which is connected to the coupling medium supply element 172.

As previously described, the control device 410 may be configured to signal various states to the user. To this end, the control device 410 may comprise one or more information display elements.

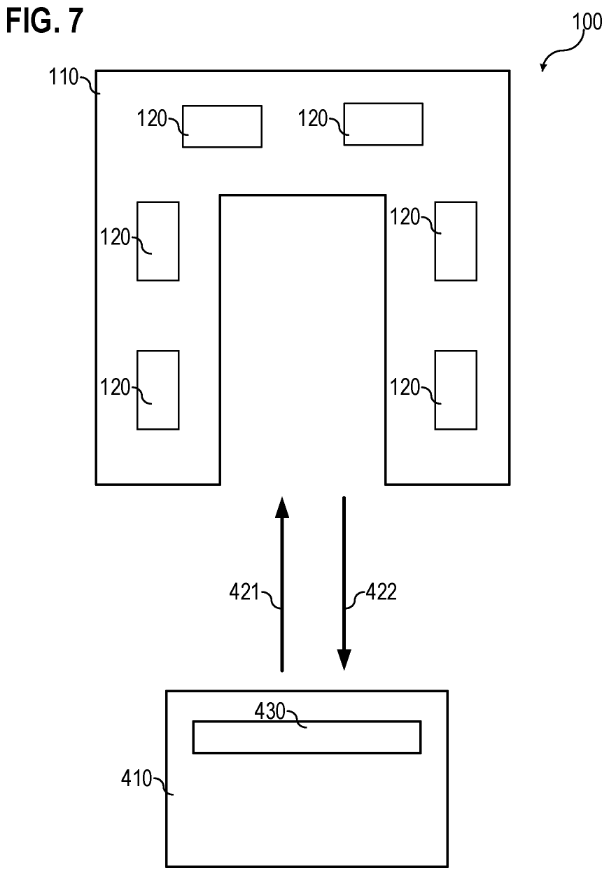

FIG. 7 schematically illustrates an ultrasound system 100 according to various aspects, comprising an information display element 430 for displaying one of one or more pieces of information.

The one or more pieces of information may comprise (e.g., may be) results of determining the one or more properties of the one or more structures within the oral cavity. The one or more pieces of information may (e.g., may be), for example, information about a fill level of an energy storage device of the ultrasound system 100. The one or more pieces of information may comprise (e.g., be), for example, information about a fill level of the coupling medium (e.g., in the coupling medium storage 171). For example, the one or more pieces of information may comprise (e.g., may be) information about a progress of the regeneration process and/or cleaning process. For example, the one or more pieces of information may comprise information about when and/or whether the cleaning process and/or regeneration process has been completed. For example, the one or more pieces of information may comprise (e.g., be) error messages.

According to various aspects, the one or more information display elements 430 may comprise (e.g., may be) one or more visual information display elements, audible information display elements, and/or haptic information display elements.

The one or more optical information display elements may, for example, be lighting elements (e.g. lamps, LED elements, etc.). For example, the one or more information display elements may comprise (e.g. be) one or more screens (e.g. displays) on which the information may be displayed.

The one or more acoustic information display elements may, for example, be sound output elements (e.g. speakers) that are configured to emit sounds. Thus, for example, an acoustic error message may be played when an error occurs. For example, the start and/or end of a cleaning process or a regeneration process may be signaled via a respective characteristic tone.

The one or more haptic information display elements may, for example, be vibration elements (e.g. vibration motors) that are configured to vibrate. Thus, for example, a haptic message may be provided by a characteristic vibration sequence when a fault occurs. For example, the start and/or end of a cleaning process or a regeneration process may be signaled via a respective characteristic vibration sequence.

According to various aspects, the bite guard 110 may also comprise one or more haptic information display elements. For example, the one or more ultrasound transducers 120 may be configured as haptic information display elements. Thus, for example, a characteristic vibration may be generated in the oral cavity that represents a certain information.

According to various aspects, the ultrasound system 100 may comprise two bite guards 110. Each of the two bite guards 110 may be/is analogous to the descriptions herein. For example, a first of the two bite guards may be adapted for a user's upper jaw and the second of the two bite guards may be adapted for a user's lower jaw. The two bite guards may be separate from each other or connected to each other. A separate design may, for example, ensure increased wearing comfort. An embodiment in which the two bite guards are connected may possibly offer a simpler application for a third party (e.g. in conjunction with a handle), which may be advantageous in particular in the region of grooming and/or in the region of animal dental hygiene. Furthermore, the connected design enables further components (such as energy storage, etc.) to be/be arranged within the connecting part.

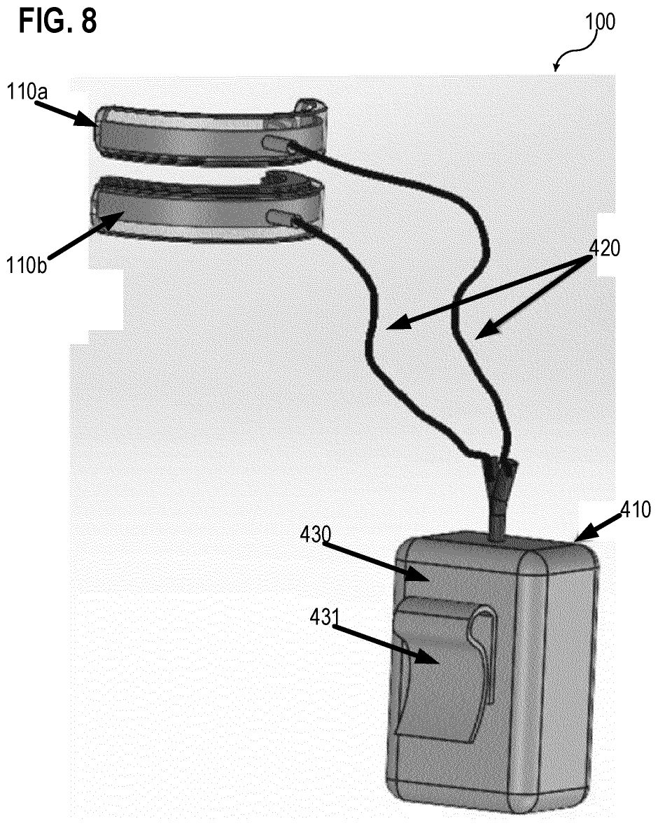

FIG. 8 schematically illustrates an ultrasound system 100 according to various aspects comprising two (separate) bite guards 110a, 110b. For example, the first bite guard 110a may be adapted for an upper jaw of a user and the second bite guard 110b may be adapted for the lower jaw of the user. Further, the ultrasound system 100 may comprise a control device 410 connected to each of the two bite guards 110a, 110b via a connecting element 420 (e.g., a cable, a cord). The connecting elements 420 may be configured for wired information transmission between the control device and the respective bite guards 110a, 110b. For example, the connecting elements 420 may also be merely connections so as not to lose the bite guards 110a, 110b. In such a case, the information transmission would be wireless (as described above).

For example, the control device 410 may comprise necessary electronic components for power supply, control, measurement data recording, and/or measurement data evaluation. For example, the control device 410 may comprise a housing 431. The housing 431 is characterized by its small geometric dimensions, a low to moderate mass (e.g. comparable to a smartphone, for example less than 500 g or for example less than 350 g or for example less than 200 g) and/or the possibility of attachment to articles of clothing of the user by means of an attachment element 431. It will be understood that a lighter control device 410 may be more easily attached to an article of clothing of a user.

For example, the bite guard 110 may be/be realized in various universal sizes, each applicable to a broad range of users, e.g., adults, children, men, women, cats, dogs, horses or other animals. The bite guard 110 may comprise a plurality of ultrasound transducers 120, e.g. ultrasound emitters or (ultrasound) actuators, and/or one or more sensors 160, each of which may be configured as described herein. For example, the plurality of ultrasound transducers 120 may be fixedly installed in the bite guard 110 (e.g., in a one bite guard cover 140). For example, the one or more sensors 160 may be permanently installed in the bite guard 110 (e.g., in a bite guard cover 140).

For example, a bite guard insert 130 may be used to customize the ultrasound system 100 to a user's bite shape. The bite guard insert 130 may comprise one or more polymers and/or one or more thermoplastic materials. For example, the bite guard insert 130 may comprise (e.g., consist of) silicone and/or ethylene vinyl acetate (EVA) and/or polymethyl methacrylate (PMMA). The bite guard insert 130 may be customized by a user to fit the shape of the user's dentition. For example, the bite guard insert 130 may be configured to be deformable when heated above a certain temperature and to retain that shape (i.e., be substantially made of a thermoplastic material) after cooling. In this case, the user may heat the bite guard insert 130 (e.g., in a water bath) and then mold the bite guard insert 130 to fit the user's tooth shape. It is understood that externally customized (e.g., by a dentist, e.g., analogous to conventional denture impressions) bite guard inserts 130 may also be used.

For example, the bite guard insert 130 may have a spacer element prior to adaptation, for example in the form of a removable layer (e.g. a sheet). After adaptation to the user's bite shape, the spacer element may be removed from the bite guard insert 130. Thus, a space may be created between the bite guard insert 130 and the user's dentition. Alternatively, the bite guard insert 130 may be configured to form a space between it and the teeth when it cools. For example, the bite guard insert 130 may also have a gap from the outset, which is not or only insignificantly (i.e. without loss of function) changed by the individual adaptation. The gap allows, for example, a coupling medium (e.g. a toothpaste) to be introduced. Thus, favorable effects may be achieved by a combination of the coupling medium and the ultrasound waves. For example, the plurality of ultrasound transducers 120 may generate ultrasound waves whereby physical effects (cavitation, mechanical friction of the cleaning bodies of the coupling medium, remineralization etc.) and/or biological effects (e.g. a stimulation for cell proliferation, a stimulation for the formation of proteins) are generated on and/or in the one or more structures within the oral cavity, which may lead to a cleaning and/or regeneration of the one or more structures within the oral cavity.

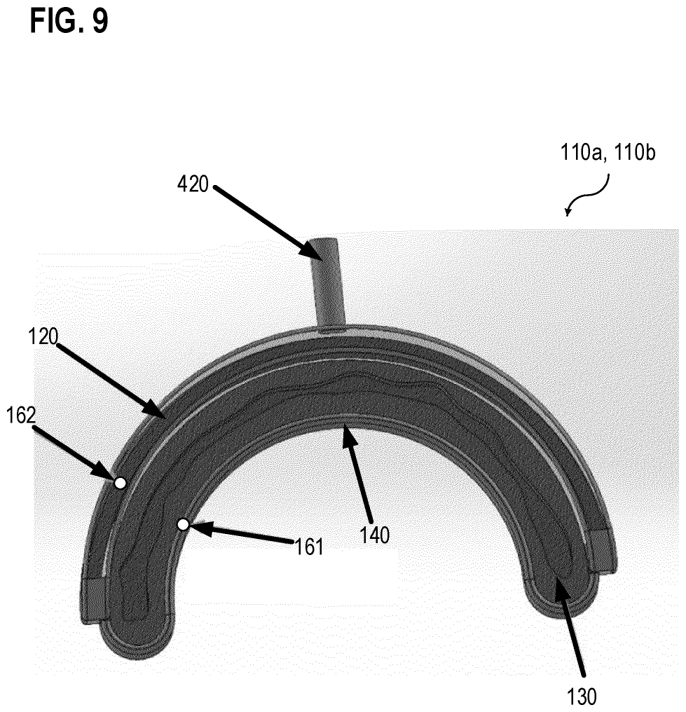

FIG. 9 illustrates an exemplary bite guard 110 of the two bite guards 110a, 110b of FIG. 8 in a view in which the bite guard insert 130 is visible. As shown, the bite guard insert 130 may be inserted into the bite guard shell. According to various aspects, the bite guard 110 may comprise one or more ultrasound transducers 120. The one or more ultrasound transducers 120 may be combined in a module. This allows, for example, rapid replacement if one of the one or more ultrasound transducers 120 is defective. Furthermore, a homogeneous ultrasound wave field may thus be generated, for example.

According to various aspects, one or more sensors 160 may be disposed in the bite guard 110. For example, one or more of the one or more sensors 160 (e.g., all of them) may each comprise a first sensor element 161 and a second sensor element 162. For example, the first sensor element 161 may be a sensor signal providing element for providing the sensor signal and the second sensor element 162 may be a measurement signal receiving element for receiving the measurement signal. As previously described, the first sensor element 161 may be an optical sensor element configured to emit light (e.g., a UV light, infrared light, visible light) comprising one or more predetermined wavelengths. The light may be transmitted and/or reflected by the one or more structures within the oral cavity and received as a measurement signal by one (or more) second sensor elements 162.

It will be understood that, although only a first sensor element 161 and a second sensor element 162 are shown as examples in FIG. 9, the bite guard 110 may comprise a plurality of first sensor elements 161 and a plurality of second sensor elements 162.

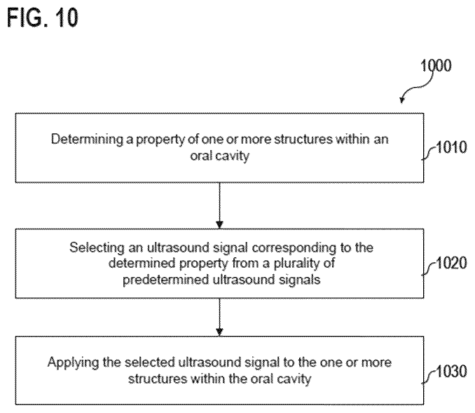

FIG. 10 illustrates an exemplary method for cleaning and/or regenerating one or more structures within the oral cavity. It will be understood that such a method may be performed using, for example, an ultrasound system 100 described herein. The method may comprise: Determining a property of the one or more structures within the oral cavity 1010. The determining may be performed, for example, using the one or more sensors 160 and the control device 410 as previously described.

Further, the method may comprise selecting an ultrasound signal corresponding to the determined property from a plurality of predetermined ultrasound signals 1020. This selection may be performed, for example, by the control device 410.

Further, the method may comprise applying the selected ultrasound signal to the one or more structures within the oral cavity 1030. For example, the control device 410 may select a suitable ultrasound signal based on the previously determined property and transmit it to the plurality of ultrasound transducers 120 within the bite guard.

According to various aspects, an ultrasound system is provided that enables non-abrasive cleaning of teeth, tooth surfaces, interdental spaces, a periodontium and structures adjacent to the teeth (directly and/or indirectly). For example, the ultrasound system may enable a cleaning effect that is not dependent on manual guidance of the tooth cleaning device, such as with a conventional toothbrush. The ultrasound system may enable a more even cleaning result than conventional toothbrushes.

According to various aspects, an ultrasound system for tooth cleaning is provided, after the use of which additional interdental care may be dispensed with.

According to various aspects, an ultrasound tooth cleaning system is provided that may facilitate tooth cleaning for people who are impaired due to their age or health.

According to various aspects, an ultrasound system for tooth cleaning is provided which may stimulate regeneration of the tooth structure, the periodontium and the jawbone.

According to various aspects, an ultrasound system for tooth cleaning is provided that may be used quietly.

The Following Describes Some Examples That Relate to What Is Described Herein and Shown in the Figures.

Example 1 is an ultrasound system that may comprise: a bite guard for insertion and positioning in an oral cavity, wherein the bite guard may comprise one or more ultrasound transducers for applying a selected ultrasound signal to one or more structures within an oral cavity for cleaning and/or regenerating the one or more structures within the oral cavity. For example, the one or more structures within the oral cavity may comprise (e.g., may be) a plurality of teeth and/or structures adjacent (e.g., directly and/or indirectly) to a plurality of teeth, such as interdental spaces corresponding to the plurality of teeth, a periodontium, and/or adjacent tissue (e.g., gums), etc.

Example 2 is an ultrasound system according to example 1, wherein the bite guard may comprise: a bite guard cover in which the one or more ultrasound transducers are positioned, and a bite guard insert which is adaptable to the one or more structures within the oral cavity and which is configured to be reversibly attached within the bite guard cover.

Example 3 is an ultrasound system according to example 2, wherein the bite guard insert may comprise a thermoplastic material configured to be adapted to the one or more structures within the oral cavity (e.g. one or more teeth) based on heating.

Example 4 is an ultrasound system according to example 2 or 3, wherein the bite guard may optionally further comprise: one or more attachment elements for reversibly attaching the bite guard insert to the bite guard cover.

Example 5 is an ultrasound system according to example 4, wherein the one or more attachment elements may comprise one or more of the following elements (e.g. may be): one or more mechanical attachment elements (e.g. plug-in elements) for mechanically attaching the bite guard insert in the bite guard cover, one or more magnetic elements for magnetically attaching the bite guard insert in the bite guard cover, and/or one or more adhesive elements for adhering the bite guard insert in the bite guard cover.

Example 6 is an ultrasound system according to any one of examples 1 to 5, wherein the one or more ultrasound transducers may comprise (e.g. be) one or more ultrasound emitters.

Example 7 is an ultrasound system according to any one of examples 1 to 6, optionally further comprising: a control device configured to select and/or provide the selected ultrasound signal, wherein the selected ultrasound signal may be selectable from a plurality of selectable ultrasound signals, and wherein the plurality of selectable ultrasound signals each differ in their respective frequency ranges, intensities and/or pulse frequencies.

Example 8 is an ultrasound system according to example 7, wherein a selectable ultrasound signal of the one or more selectable ultrasound signals may be suitable for stimulating a regeneration (e.g. a tissue regeneration of the gums, a tissue regeneration of the bone tissue, a cell proliferation, a remineralization of the teeth), and wherein, for example, a selectable ultrasound signal for stimulating a regeneration is configured to generate ultrasound waves in a HIFU region and/or in a LIPUS region.

Ultrasound waves from the HIFU region may have frequencies between 0.5 MHz and 10 MHz, for example between 1 MHz and 5 MHz. Ultrasound waves from the HIFU region may have a power of between 0.5 kW/cm² and 20 kW/cm², for example between 1 kW/cm² and 10 kW/cm² or for example of around 5 kW/cm² (e.g. with a total irradiation time of less than 3 seconds). Due to such a high energy density and the associated temperature increase, ultrasound waves from the HIFU region should only be applied over very short periods of time (e.g. less than 1 ms, for example less than 10 μs or for example less than 1 μs). Ultrasound waves from the LIPUS region may have frequencies between 0.5 MHz and 2 MHz, for example between 1 MHz and 1.5 MHz. Ultrasound waves from the LIPUS region may have a power between 1 mW/cm² and 1000 mW/cm², for example between 10 mW/cm² and 750 mW/cm² or, for example, between 20 mW/cm² and 500 mW/cm². Ultrasound waves from the LIPUS region may have a pulse frequency between 0.1 kHz and 10 kHz, for example between 0.5 kHz and 5 kHz or, for example, of 1 kHz. Ultrasound waves from the LIPUS region may have a pulse duration of between 1 μs and 1 ms, for example between 50 μs and 500 μs or, for example, of around 200 μs.

Example 9 is an ultrasound system according to example 7 or 8, wherein a selectable ultrasound signal of the one or more selectable ultrasound signals may be adapted to remove contaminants from one or more surfaces of the one or more structures within the oral cavity (e.g. the plurality of teeth and/or from/of the structures adjacent to the plurality of teeth (e.g. the corresponding interdental spaces), and wherein, for example, a selectable ultrasound signal for removing contaminants may have a frequency in the range between 1 kHz and 20 MHz (e.g. between 10 kHz and 7 MHz). the corresponding interdental spaces)), and wherein, for example, a selectable ultrasound signal for removing contaminants may have a frequency in the region between 1 kHz and 20 MHz (for example, between 10 kHz and 7 MHz or, for example, between 18 kHz and 5 MHz), may have an intensity between 0.01 W/cm² and 1 kW/cm² (for example between 0.05 W/cm² and 500 W/cm² or for example between 0.1 W/cm² and 100 W/cm²), and may have a pulse frequency between 10 Hz and 20 kHz (for example between 100 Hz and 10 kHz or for example between 500 Hz and 5 kHz).

Example 10 is an ultrasound system according to any one of examples 7 to 9, wherein a selectable ultrasound signal of the one or more selectable ultrasound signals may be adapted to remove tartar from one or more surfaces of the one or more structures within the oral cavity (e.g. from one or more teeth), and wherein, for example, a selectable ultrasound signal for removing tartar may have a frequency in the region between 1 kHz and 20 MHz (for example between 10 kHz and 15 MHz or for example between 18 kHz and 10 MHz), an intensity between 0,01 W/cm² and 10 kW/cm² (for example between 0.05 W/cm² and 5 kW/cm² or for example between 0.1 W/cm² and 1 kW/cm²), and may have a pulse frequency between 10 Hz and 20 kHz (for example between 100 Hz and 10 kHz or for example between 500 Hz and 5 kHz).

Example 11 is an ultrasound system according to any one of examples 7 to 10, wherein the selection of the selected ultrasound signal may be based on an input from a user of the ultrasound system.

Example 12 is an ultrasound system according to any one of examples 7 to 11, wherein the bite guard may comprise one or more sensors for determining a property (e.g. a type of contamination and/or a type of pathological change) of the one or more structures within the oral cavity; and wherein the selected ultrasound signal may be associated with the determined property of the one or more structures within the oral cavity.

Example 13 is an ultrasound system according to example 12, wherein the control device may optionally further be configured to determine the property of the one or more structures within the oral cavity by means of the one or more sensors, and wherein the control device may optionally further be configured to select and provide the ultrasound signal based on the determined property.

Example 14 is an ultrasound system according to example 13, wherein the control device may optionally further be configured to increase an intensity and/or to decrease an intensity of the selected ultrasound signal depending on a characteristic (e.g. a presence, a degree, etc.) of the determined property of the one or more structures within the oral cavity.

Example 15 is an ultrasound system according to example 12 or 13, wherein the control device may optionally further be configured to start and/or stop the selected ultrasound signal depending on a presence or an absence of the determined property of the one or more structures within the oral cavity.

Example 16 is an ultrasound system according to any one of examples 13 to 15, wherein the property of the one or more structures within the oral cavity may be a property of the following: a contamination (e.g., presence, type of contamination, degree of contamination) of the one or more structures within the oral cavity (e.g., of the one or more teeth and/or the interdental spaces corresponding thereto), or one or more pathological changes of the one or more structures within the oral cavity (e.g., of the one or more teeth and/or the interdental spaces corresponding thereto). e.g. of the multiple teeth and/or the corresponding interdental spaces), or one or more pathological changes in the one or more structures within the oral cavity (e.g. of one or more teeth (e.g. caries, tartar, defects, demineralization), of the gums (e.g. inflammation, tumors), etc.).

Example 17 is an ultrasound system according to any one of examples 12 to 15, wherein the one or more sensors may comprise (e.g. be) optical sensors, and wherein the one or more sensors may be configured, for example, as optical transmission sensors and/or optical reflection sensors.

Example 18 is an ultrasound system according to any one of examples 12 to 17, wherein the one or more sensors may comprise: one or more sensor signal providing elements (e.g. light emitting diodes) for providing respective predetermined sensor signals, and one or more measurement signal receiving elements (e.g. photodetectors) for receiving measurement signals, wherein a deviation of the measurement signal from the corresponding predetermined sensor signal may represent a property of the one or more structures within the oral cavity.

Example 19 is an ultrasound system according to any one of examples 1 to 18, wherein the ultrasound system, for example the bite guard (e.g. the bite guard insert and/or the bite guard cover), may be bristle-free. For example, the cleaning effect and/or the transmission of ultrasound may be achieved entirely without bristles.

Example 20 is an ultrasound system according to any one of examples 1 to 19, wherein the bite guard comprises a coupling medium inlet and/or a coupling medium storage for an ultrasound coupling medium for applying the ultrasound signal to the one or more structures within the oral cavity.

Example 21 is an ultrasound system according to any one of examples 7 to 20, optionally further comprising: one or more information display elements for displaying one of the one or more pieces of information, wherein the control device may be configured to display a selected one of the one or more pieces of information by means of the one or more information display elements.

Example 22 is an ultrasound system according to example 21, wherein displaying the selected information may comprise vibrating one or more components of the ultrasound system, wherein the vibration is representative of the information to be displayed. For example, the information display element may comprise one or more vibrating elements. For example, a group of the plurality of ultrasound transducers may also function as vibrating elements. For example, a specific vibration (e.g. a vibration intensity, a vibration sequence, a vibration frequency) may be associated with each of the one or more states.

Example 23 is an ultrasound system according to example 21 or 22, wherein the displaying of the selected information may comprise: Playing one or more tones, wherein the one or more tones are representative of the information to be displayed. For example, the information display element may comprise one or more speakers. For example, one or more specific tones (e.g. a pitch, a tone sequence (e.g. a melody), a volume) may be associated with each of the one or more states.

Example 24 is an ultrasound system according to any one of examples 21 to 23, wherein displaying the selected information may comprise Displaying an optical signal, wherein the optical signal is representative of the information to be displayed. For example, the information display element may comprise one or more luminous elements (e.g. LEDs, lamps) and/or a display (e.g. a screen). For example, a specific optical signal (e.g. a pictogram, a specific color, switching on/off of one or more light elements, a color sequence (e.g. by means of several light-emitting diodes), etc.) may be assigned to each of the one or more states.

Example 25 is an ultrasound system according to any one of examples 19 to 22, wherein the selected information represents one or more of the following information: the selected ultrasound signal (e.g. which ultrasound signal has been/is selected), and/or an intensity of the selected ultrasound signal, and/or whether the selected ultrasound signal is/will be applied, and/or whether the ultrasound system is on or off, and/or a progress of a cleaning process, and/or a progress of a regeneration process, and/or errors, faults, other indications of the ultrasound system.

Example 26 is an ultrasound system according to any one of examples 7 to 25, optionally further comprising: an energy source configured to provide an energy for operating the ultrasound system.

Example 27 is an ultrasound system according to any one of examples 1 to 26, wherein the one or more structures within the oral cavity may comprise one or more teeth (e.g. including surfaces thereof), and wherein the one or more structures within the oral cavity may comprise, for example, the interdental spaces corresponding to the plurality of teeth.

Example 28 is an ultrasound system comprising: a control device configured to select and/or provide a selected ultrasound signal selectable from a plurality of selectable ultrasound signals differing in their respective frequency ranges, intensities and/or pulse frequencies, a bite guard for insertion and positioning within an oral cavity, wherein the bite guard comprises one or more ultrasound transducers for applying a selected ultrasound signal to one or more structures within an oral cavity for cleaning and/or regenerating the one or more structures within the oral cavity; wherein the bite guard further comprises one or more optical sensors for determining a property of the one or more structures within the oral cavity; and wherein the control device is configured to select and/or provide the selected ultrasound signal based on the determined property of the one or more structures within the oral cavity.

The features described in connection with the ultrasound systems of examples 1 to 27 may also be applied in an analogous manner to the ultrasound system of example 28, and vice versa.

Example 29 is a use of an ultrasound system according to any one of examples 1 to 28 for cleaning and/or regenerating one or more structures within the oral cavity.

Example 30 is a method of cleaning and/or regenerating one or more structures within an oral cavity, the method may comprising: determining a property of the one or more structures within the oral cavity; selecting an ultrasound signal corresponding to the determined property from a plurality of predetermined ultrasound signals; and applying the selected ultrasound signal to the one or more structures within the oral cavity.

Example 31 is a method according to example 30, optionally further comprising: re-determining the determined property; comparing the re-determined property to the previously determined property; continuing to apply the selected ultrasound signal to the one or more structures within the oral cavity if the comparison of the properties indicates that the re-determined property differs from the previously determined property by less than a predetermined threshold; and stopping application of the selected ultrasound signal to the one or more structures within the oral cavity if the comparison indicates that the redetermined property differs from the previously determined property by at least the predetermined limit.

Example 32 is a method according to example 30 or 31, optionally further comprising: starting a counter with the application of the selected ultrasound signal to the one or more structures within the oral cavity, and stopping the application of the selected ultrasound signal to the one or more structures within the oral cavity when the counter reaches a predetermined value.

Example 33 is a method according to example 32, wherein the counter comprises (e.g., is) a clock (e.g., a stopwatch).

Example 34 is a method according to example 32 or 33, wherein the counter (e.g. optionally further) is configured to count how many times the determined property has been counted again.

Example 35 is a method according to any one of examples 30 to 34, wherein the one or more structures within the oral cavity may comprise one or more teeth (e.g., including surfaces thereof), and wherein the one or more structures within the oral cavity may comprise, for example, the interdental spaces corresponding to the plurality of teeth.

Example 36 is a method according to any one of examples 30 to 35, wherein the determining of a property of the one or more structures within the oral cavity is performed by means of one or more sensors, for example optical sensors.

For example, the methods of examples 30 to 35 may be performed by means of an ultrasound system according to any one of examples 1 to 28. For example, the described control device of the ultrasound system of examples 1 to 28 may be configured to perform one or more of the methods according to examples 30 to 36.

For example, the features respectively described in connection with the ultrasound systems of examples 1 to 28 may also be transferred to the methods of examples 30 to 36 and vice versa.

1. An ultrasound system comprising:
a bite guard for insertion and positioning in an oral cavity,
wherein the bite guard comprises one or more ultrasound transducers for applying a selected ultrasound signal to one or more structures within an oral cavity for cleaning and/or regenerating the one or more structures within the oral cavity.

2. The ultrasound system according to claim 1, further comprising:
a control device configured to select and/or provide a selected ultrasound signal, selectable from a plurality of selectable ultrasound signals differing in their respective frequency ranges, intensities and/or pulse frequencies;
wherein the bite guard further comprises one or more optical sensors for determining a property of the one or more structures within the oral cavity; and
wherein the control device is configured to select and/or provide the selected ultrasound signal based on the determined property of the one or more structures within the oral cavity.

3. The ultrasound system according to claim 1, wherein the bite guard comprises:
a bite guard cover in which the one or more ultrasound transducers are positioned, and
a bite guard insert which is adaptable to the one or more structures within the oral cavity and which is configured to be reversibly attached within the bite guard cover.

4. The ultrasound system according to claim 3, wherein
the bite guard insert comprises a thermoplastic material such that it can be adapted to the one or more structures within the oral cavity upon heating.

5. The ultrasound system according to claim 3, wherein the bite guard further comprises:
one or more attachment elements for reversibly attaching the bite guard insert in the bite guard cover,
wherein the one or more attachment elements comprise one or more of the following: mechanical attachment elements for mechanically attaching the bite guard insert in the bite guard cover, magnetic elements for magnetically attaching the bite guard insert in the bite guard cover, and/or adhesive elements for adhering the bite guard insert in the bite guard cover.

6. The ultrasound system according to claim 1,
wherein the one or more ultrasound transducers comprise one or more ultrasound emitters.

7. The ultrasound system according to claim 1, or further comprising:
a control device configured to select and/or provide the selected ultrasound signal,
whereby the selected ultrasound signal can be chosen from several selectable ultrasound signals which differ in their respective frequency ranges, intensities and/or pulse frequencies.

8. The ultrasound system according to claim 2,
wherein a selectable ultrasound signal of the one or more selectable ultrasound signals is adapted to stimulate regeneration, and
wherein a selectable ultrasound signal for stimulating regeneration is configured to generate ultrasound waves in a HIFU region and/or a LIPUS region.

9. The ultrasound system according to claim 2,
wherein a selectable ultrasound signal of the one or more selectable ultrasound signals is adapted to remove contaminants from one or more surfaces of the one or more structures within the oral cavity, and
wherein a selectable ultrasound signal for removing impurities comprises a frequency between 1 kHz and 20 MHz, an intensity between 0.01 W/cm² and 1 kW/cm², and a pulse frequency between 10 Hz and 20 kHz.

10. The ultrasound system according to claim 2,
wherein a selectable ultrasound signal of the one or more selectable ultrasound signals is adapted to remove tartar from one or more surfaces of the one or more structures within the oral cavity, and
wherein a selectable ultrasound signal for removing tartar has a frequency between 1 kHz and 20 MHz, has an intensity between 0.01 W/cm² and 10 kW/cm², and has a pulse frequency between 10 Hz and 20 kHz.

11. The ultrasound system according to claim 7,
wherein the bite guard comprises one or more sensors for determining a property of the one or more structures within the oral cavity; and
wherein the selected ultrasound signal is associated with the determined property of the one or more structures within the oral cavity.

12. The ultrasound system according to claim 2,
wherein the control device is further configured to determine the property of the one or more structures within the oral cavity by means of the one or more optical sensors and
wherein the control device is further configured to select and provide the selected ultrasound signal based on the determined property.

13. The ultrasound system according to claim 12,
wherein the control device is further configured to increase an intensity and/or decrease the intensity of the selected ultrasound signal depending on a characteristic of the determined property of the one or more structures within the oral cavity; and
wherein the control device further configured to start and/or stop the selected ultrasound signal depending on a presence or an absence of the determined property of the one or more structures within the oral cavity.

14. The ultrasound system according to claim 1,
wherein the bite guard is bristle-free.

15. (canceled)

16. A method of cleaning and/or regenerating one or more structures within an oral cavity, the method comprising:
determining a property of the one or more structures within the oral cavity;
selecting an ultrasound signal corresponding to the determined property from a plurality of predetermined ultrasound signals; and
applying the selected ultrasound signal to the one or more structures within the oral cavity.

17. The method according to claim 16, wherein the determining a property of the one or more structures within the oral cavity is performed by optical sensors.
